(19) United States
(12) Patent Application Publication
(10) Pub. No.: US 2012/0030547 A1
(43) Pub. Date: Feb. 2, 2012

(54) SYSTEM AND METHOD FOR SAVING BATTERY POWER IN A VITAL-SIGNS MONITOR

(75) Inventors: Mark Raptis, Valley Center, CA (US); Amir Jafri, San Diego, CA (US); Alison Burdett, Oxford (GB); Ganesh Kathiresan, Osterley (GB)

(73) Assignee: CareFusion 303, Inc., San Diego, CA (US)

(21) Appl. No.: 12/844,789

(22) Filed: Jul. 27, 2010

(51) Int. Cl. H03M 13/09; G06F 15/16; G06F 11/10; A61B 5/00

(52) U.S. Cl. 714/799; 600/301; 709/204; 714/E11.032

(57) ABSTRACT

A vital-signs device in a patient monitoring system is disclosed. The patch includes a housing configured to be attached to the skin of a patient. The housing contain monitoring circuitry configured to acquire and store measurements of vital signs of the patient, a wireless transmitter configured to transmit signals to another device, a wireless receiver configured to receive signals from the other device; and a processor operably connected to the monitoring circuitry, transmitter, and receiver. Upon receipt of an upload signal from the other device, the processor is configured to send a message to the other device via the transmitter. The message packet structure includes a data payload of variable size, a header containing transmit and route information and data payload length, and a data integrity check value.

### CROSS-REFERENCES TO RELATED APPLICATIONS

The following applications disclose certain common subject matter with the present application: A Vital-Signs Monitor with Encapsulation Arrangement, U.S. application Ser. No. 12/844; A Vital-Signs Monitor with Spaced Electrodes, U.S. application Ser. No. 12/844,769; A Vital-Signs Patch Having a Strain Relief, U.S. application Ser. No. 12/844,774; A Temperature Probe Suitable for Axillary Reading, U.S. application Ser. No. 12/844,775; System and Method for Monitoring Body Temperature of a Person, U.S. application Ser. No. 12/844,771; A System and Method for Storing and Forwarding Data from a Vital-Signs Monitor, U.S. application Ser. No. 12/844,780; A System and Method for Conserving Battery Power in a Patient Monitoring System, U.S. application Ser. No. 12/844,796; A System and Method for Saving Battery Power in a Patient Monitoring System, U.S. application Ser. No. 12/844,801; A System And Method for Tracking Vital-Signs Monitor Patches, U.S. application Ser. No. 12/844,788; A System And Method for Reducing False Alarms Associated with Vital-Signs Monitoring, U.S. application Ser. No. 12/844,794; A System And Method for Location Tracking of Patients in a Vital-Signs Monitoring System, U.S. application Ser. No. 12/844,781; A System And Method for Reducing False Alarms Based on Motion and Location Sensing, U.S. application Ser. No. 12/844,765; all of the listed applications filed on Jul. 27, 2010.

### BACKGROUND

1. Field

The present disclosure generally relates to systems and methods of physiological monitoring, and, in particular, relates to monitoring of vital signs of patients.

2. Description of the Related Art

Some of the most basic indicators of a person's health are those physiological measurements that reflect basic body functions and are commonly referred to as a person's “vital signs.” The four measurements commonly considered to be vital signs are body temperature, pulse rate, blood pressure, and respiratory rate. Some clinicians consider oxygen saturation (S₀₂) to be a “fifth vital sign” particularly for pediatric or geriatric cases. Some or all of these measurements may be performed routinely upon a patient when they arrive at a healthcare facility, whether it is a routine visit to their doctor or arrival at an Emergency Room (ER).

Vital signs are frequently taken by a nurse using basic tools including a thermometer to measure body temperature, a sphygmomanometer to measure blood pressure, and a watch to count the number of breaths or the number of heart beats in a defined period of time which is then converted to a “per minute” rate. If a patient's pulse is weak, it may not be possible to detect a pulse by hand and the nurse may use a stethoscope to amplify the sound of the patient's heart beat so that she can count the beats. Oxygen saturation of the blood is most easily measured with a pulse oximeter.

When a patient is admitted to a hospital, it is common for vital signs to be measured and recorded at regular intervals during the patient's stay to monitor their condition. A typical interval is 4 hours, which leads to the undesirable requirement for a nurse to awaken a patient in the middle of the night to take vital sign measurements.

When a patient is admitted to an ER, it is common for a nurse to do a “triage” assessment of the patient's condition that will determine how quickly the patient receives treatment. During busy times in an ER, a patient who does not appear to have a life-threatening injury may wait for hours until more-serious cases have been treated. While the patient may be reassessed at intervals while awaiting treatment, the patient may not be under observation between these reassessments.

Measuring certain vital signs is normally intrusive at best and difficult to do on a continuous basis. Measurement of body temperature, for example, is commonly done by placing an oral thermometer under the tongue or placing an infrared thermometer in the ear canal such that the tympanic membrane, which shared blood circulation with the brain, is in the sensor's field of view. Another method of taking a body temperature is by placing a thermometer under the arm, referred to as an “axillary” measurement as axilla is the Latin word for armpit. Skin temperature can be measured using a stick-on strip that may contain panels that change color to indicate the temperature of the skin below the strip.

Measurement of respiration is easy for a nurse to do, but relatively complicated for equipment to achieve. A method of automatically measuring respiration is to encircle the upper torso with a flexible band that can detect the physical expansion of the rib cage when a patient inhales. An alternate technique is to measure a high-frequency electrical impedance between two electrodes placed on the torso and detect the change in impedance created when the lungs fill with air. The electrodes are typically placed on opposite sides of one or both lungs, resulting in placement on the front and back or on the left and right sides of the torso, commonly done with adhesive electrodes connected by wires or by using a torso band with multiple electrodes in the strap.

Measurement of pulse is also relatively easy for a nurse to do and intrusive for equipment to achieve. A common automatic method of measuring a pulse is to use an electrocardiograph (ECG or EKG) to detect the electrical activity of the heart. An EKG machine may use 12 electrodes placed at defined points on the body to detect various signals associated with the heart function. Another common piece of equipment is simply called a “heart rate monitor.” Widely sold for use in exercise and training, heart rate monitors commonly consist of a torso band, in which are embedded two electrodes held against the skin and a small electronics package. Such heart rate monitors can communicate wirelessly to other equipment such as a small device that is worn like a wristwatch and that can transfer data wirelessly to a PC.

Nurses are expected to provide complete care to an assigned number of patients. The workload of a typical nurse is increasing, driven by a combination of a continuing shortage of nurses, an increase in the number of formal procedures that must be followed, and an expectation of increased documentation. Replacing the manual measurement and logging of vital signs with a system that measures and records vital signs would enable a nurse to spend more time on other activities and avoid the potential for error that is inherent in any manual procedure.

### SUMMARY

For some or all of the reasons listed above, there is a need to be able to continuously monitor patients in different settings. In addition, it is desirable for this monitoring to be done with limited interference with a patient's mobility or interfering with their other activities.

Embodiments of the patient monitoring system disclosed herein measure certain vital signs of a patient, which include respiratory rate, pulse rate, blood pressure, body temperature, and, in some cases, oxygen saturation (S_{O2}), on a regular basis and compare these measurements to defined limits.

In certain aspects of the present disclosure, a patch that is part of a patient monitoring system is disclosed according to certain embodiments. The patch contains a housing that is configured to be attached to the skin of a patient. The housing contains circuitry that acquires and stores measurements of the vital signs of the patient, a transmitter and a receiver, and a processor. Upon receipt of an ‘upload’ command from another device, the processor obtains data from the monitoring circuitry and sends a message with a structure that includes a data payload, a message header containing transmit and route information the data payload length, and a data integrity check value.

In certain aspects of the present disclosure, a patient monitoring system is disclosed according to certain embodiments. The system includes a vital-signs patch configured to be attached to the skin of a patient and a bridge that communicates with the patch using a message packet structure. The message packet structure contains a header containing transmit and route information, and data payload length, a data payload, and a data integrity check value. The data payload length is variable. The bridge initiates each message exchange with a command message and the patch responds with an acknowledgement message.

In certain aspects of the present disclosure, a method of conserving battery power is disclosed according to certain embodiments. The method includes the steps of receiving a transmit signal, retrieving vital-signs data, creating a message that contains a data payload, a header, and a data integrity check value, and transmitting the message. The data payload contains at least a portion of the vital-signs data. The header contains transmission and routing information and the data payload length.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 is a diagram illustrating an exemplary embodiment of a patient monitoring system according to certain aspects of the present disclosure.

FIG. 2A is a perspective view of the vital-signs monitor patch of FIG. 1 according to certain aspects of the present disclosure.

FIG. 2B is a cross-section of the vital-signs monitor patch of FIG. 1 according to certain aspects of the present disclosure.

FIG. 2C is a functional block diagram illustrating exemplary electronic and sensor components of the vital-signs monitor patch of FIG. 1 according to certain aspects of the present disclosure.

FIG. 3A is a functional schematic diagram of an embodiment of the bridge according to certain aspects of the present disclosure.

FIG. 3B is a functional schematic diagram of an embodiment of the surveillance server according to certain aspects of the present disclosure.

FIGS. 4A & 4B illustrate Ethernet communication protocol 802.11b and an associated message structure.

FIG. 5 discloses an embodiment of a communication protocol according to certain aspects of the present disclosure.

### DETAILED DESCRIPTION

Periodic monitoring of patients in a hospital is desirable at least to ensure that patients do not suffer an un-noticed sudden deterioration in their condition or a secondary injury during their stay in the hospital. It is impractical to provide continuous monitoring by a clinician and cumbersome to connect sensors to a patient, which are then connected to a fixed monitoring instrument by wires. Furthermore, systems that sound an alarm when the measured value exceeds a threshold value may sound alarms so often and in situations that are not truly serious that such alarms are ignored by clinicians.

Measuring vital signs is difficult to do on a continuous basis. Accurate measurement of cardiac pulse, for example, can be done using an electrocardiograph (ECG or EKG) to detect the electrical activity of the heart. An EKG machine may use up to 12 electrodes placed at various points on the body to detect various signals associated with the cardiac function. Another common piece of equipment is termed a “heart rate monitor.” Widely sold for use in exercise and physical training, heart rate monitors may comprise a torso band in which are embedded two electrodes held against the skin and a small electronics package. Such heart rate monitors can communicate wirelessly to other equipment such as a small device that is worn like a wristwatch and that can transfer data wirelessly to a personal computer (PC).

Monitoring of patients that is referred to as “continuous” is frequently periodic, in that measurements are taken at intervals. In many cases, the process to make a single measurement takes a certain amount of time, such that even back-to-back measurements produce values at an interval equal to the time that it takes to make the measurement. For the purpose of vital sign measurement, a sequence of repeated measurements can be considered to be “continuous” when the vital sign is not likely to change an amount that is of clinical significance within the interval between measurements. For example, a measurement of blood pressure every 10 minutes may be considered “continuous” if it is considered unlikely that a patient's blood pressure can change by a clinically significant amount within 10 minutes. The interval appropriate for measurements to be considered continuous may depend on a variety of factors including the type of injury or treatment and the patient's medical history. Compared to intervals of 4-8 hours for manual vital sign measurement in a hospital, measurement intervals of 30 minutes to several hours may still be considered “continuous.”

Certain exemplary embodiments of the present disclosure include a system that comprises a vital-signs monitor patch that is attached to the patient, and a bridge that communicates with monitor patches and links them to a central server that processes the data, where the server can send data and alarms to a hospital system according to algorithms and protocols defined by the hospital.

The construction of the vital-signs monitor patch is described according to certain aspects of the present disclosure. As the patch may be worn continuously for a period of time that may be several days, as is described in the following disclosure, it is desirable to encapsulate the components of the patch such that the patient can bathe or shower and engage in their normal activities without degradation of the patch function. An exemplary configuration of the construction of the patch to provide a hermetically sealed enclosure about the electronics is disclosed.

In the following detailed description, numerous specific details are set forth to provide a full understanding of the present disclosure. It will be apparent, however, to one ordinarily skilled in the art that embodiments of the present disclosure may be practiced without some of the specific details. In other instances, well-known structures and techniques have not been shown in detail so as not to obscure the disclosure.

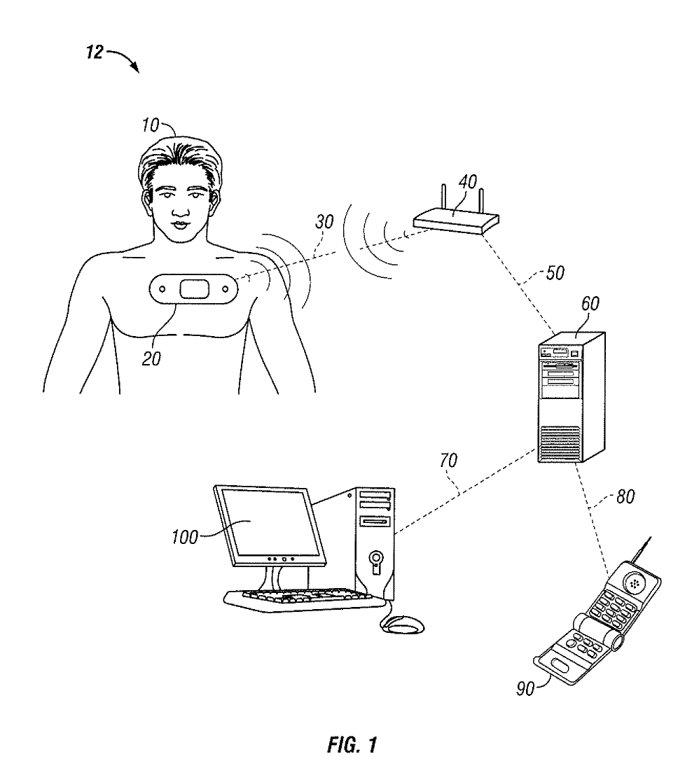

FIG. 1 discloses a vital sign monitoring system according to certain embodiments of the present disclosure. The vital sign monitoring system 12 includes vital-signs monitor patch 20, bridge 40, and surveillance server 60 that can send messages or interact with peripheral devices exemplified by mobile device 90 and workstation 100.

Monitor patch 20 resembles a large adhesive bandage and is applied to a patient 10 when in use. It is preferable to apply the monitor patch 20 to the upper chest of the patient 10 although other locations may be appropriate in some circumstances. Monitor patch 20 incorporates one or more electrodes (not shown) that are in contact with the skin of patient 10 to measure vital signs such as cardiac pulse rate and respiration rate. Monitor patch 20 also may include other sensors such as an accelerometer, temperature sensor, or oxygen saturation sensor to measure other characteristics associated with the patient. These other sensors may be internal to the monitor patch 20 or external sensors that are operably connected to the monitor patch 20 via a cable or wireless connection. Monitor patch 20 also includes a wireless transmitter that can both transmit and receive signals. This transmitter is preferably a short-range, low-power radio frequency (RF) device operating in one of the unlicensed radio bands. One band in the United States (US) is, for example, centered at 915 MHz and designated for industrial, scientific and medical (ISM) purposes. An example of an equivalent band in the European Union (EU) is centered at 868 MHz. Other frequencies of operation may be possible dependent upon the International Telecommunication Union (ITU), local regulations and interference from other wireless devices.

Surveillance server 60 may be a standard computer server connected to the hospital communication network and preferably located in the hospital data center or computer room, although other locations may be employed. The server 60 stores and processes signals related to the operation of the patient monitoring system 12 disclosed herein including the association of individual monitor patches 20 with patients 10 and measurement signals received from multiple monitor patches 20. Hence, although only a single patient 10 and monitor patch 20 are depicted in FIG. 1, the server 60 is able to monitor the monitor patches 20 for multiple patients 10.

Bridge 40 is a device that connects, or “bridges”, between monitor patch 20 and server 60. Bridge 40 communicates with monitor patch 20 over communication link 30 operating, in these exemplary embodiments, at approximately 915 MHz and at a power level that enables communication link 30 to function up to a distance of approximately 10 meters. It is preferable to place a bridge 40 in each room and at regular intervals along hallways of the healthcare facility where it is desired to provide the ability to communicate with monitor patches 20. Bridge 40 also is able to communicate with server 60 over network link 50 using any of a variety of computer communication systems including hardwired and wireless Ethernet using protocols such as 802.11a/b/g or 802.3af. As the communication protocols of communication link 30 and network link 50 may be very different, bridge 40 provides data buffering and protocol conversion to enable bidirectional signal transmission between monitor patch 20 and server 60.

While the embodiments illustrated by FIG. 1 employ a bridge 20 to provide communication link between the monitor patch 20 and the server 60, in certain alternative embodiments, the monitor patch 20 may engage in direct wireless communication with the server 60. In such alternative embodiments, the server 60 itself or a wireless modem connected to the server 60 may include a wireless communication system to receive data from the monitor patch 20.

In use, a monitor patch 20 is applied to a patient 10 by a clinician when it is desirable to continuously monitor basic vital signs of patient 10 while patient 10 is, in this embodiment, in a hospital. Monitor patch 20 is intended to remain attached to patient 10 for an extended period of time, for example, up to 5 days in certain embodiments, limited by the battery life of monitor patch 20. In some embodiments, monitor patch 20 is disposable when removed from patient 10.

Server 60 executes analytical protocols on the measurement data that it receives from monitor patch 20 and provides this information to clinicians through external workstations 100, preferably personal computers (PCs), laptops, or smart phones, over the hospital network 70. Server 60 may also send messages to mobile devices 90, such as cell phones or pagers, over a mobile device link 80 if a measurement signal exceeds specified parameters. Mobile device link 80 may include the hospital network 70 and internal or external wireless communication systems that are capable of sending messages that can be received by mobile devices 90.

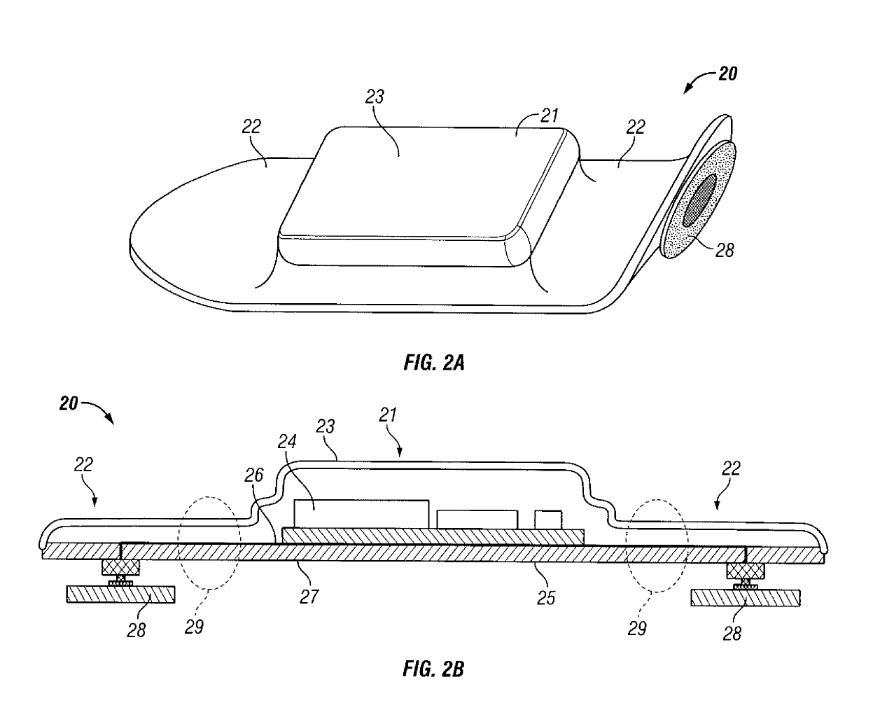

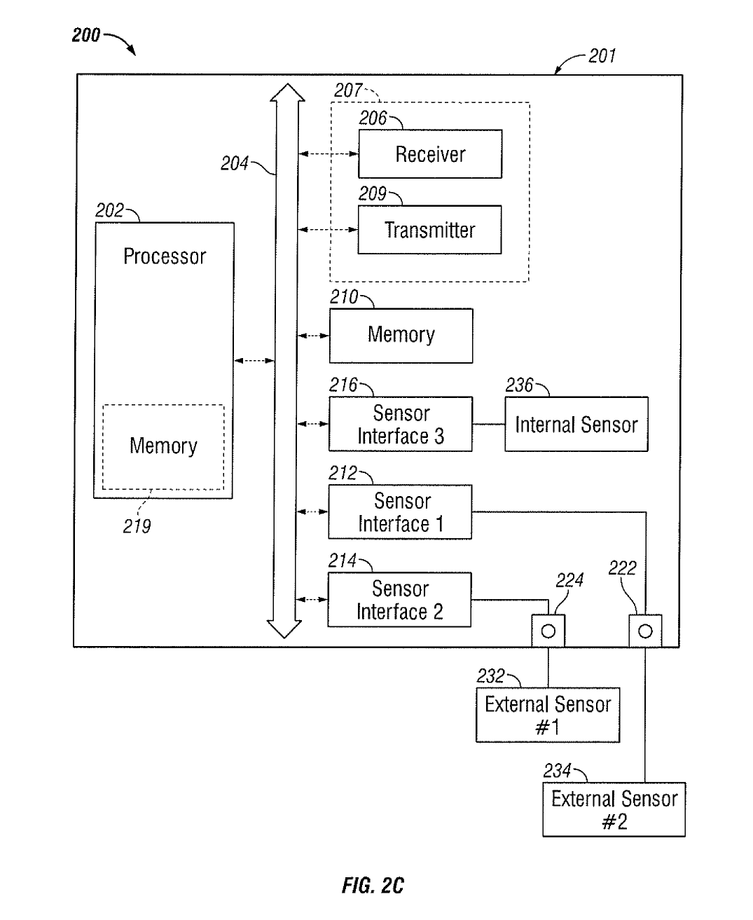

FIG. 2A is a perspective view of the vital-signs monitor patch 20 shown in FIG. 1 according to certain aspects of the present disclosure. In the illustrated embodiment, the monitor patch 20 includes component carrier 23 comprising a central segment 21 and side segments 22 on opposing sides of the central segment 21. In certain embodiments, the central segment 21 is substantially rigid and includes a circuit assembly (24, FIG. 2B) having electronic components and battery mounted to a rigid printed circuit board (PCB). The side segments 22 are flexible and include a flexible conductive circuit (26, FIG. 2B) that connect the circuit assembly 24 to electrodes 28 disposed at each end of the monitor patch 20, with side segment 22 on the right shown as being bent upwards for purposes of illustration to make one of the electrodes 28 visible in this view.

FIG. 2B is a cross-sectional view of the vital-signs patch 20 shown in FIGS. 1 and 2A according to certain aspects of the present disclosure. The circuit assembly 24 and flexible conductive circuit 26 described above can be seen herein. The flexible conductive circuit 26 operably connects the circuit assembly 24 to the electrodes 28. Top and bottom layers 23 and 27 form a housing 25 that encapsulate circuit assembly 28 to provide a water and particulate barrier as well as mechanical protection. There are sealing areas on layers 23 and 27 that encircles circuit assembly 28 and is visible in the cross-section view of FIG. 2B as areas 29. Layers 23 and 27 are sealed to each other in this area to form a substantially hermetic seal. Within the context of certain aspects of the present disclosure, the term ‘hermetic’ implies that the rate of transmission of moisture through the seal is substantially the same as through the material of the layers that are sealed to each other, and further implies that the size of particulates that can pass through the seal are below the size that can have a significant effect on circuit assembly 24. Flexible conductive circuit 26 passes through portions of sealing areas 29 and the seal between layers 23 and 27 is maintained by sealing of layers 23 and 27 to flexible circuit assembly 28. The layers 23 and 27 are thin and flexible, as is the flexible conductive circuit 26, allowing the side segment 22 of the monitor patch 20 between the electrodes 28 and the circuit assembly 24 to bend as shown in FIG. 2A.

FIG. 2C is a functional block diagram 200 illustrating exemplary electronic and sensor components of the monitor patch 20 of FIG. 1 according to certain aspects of the present disclosure. The block diagram 200 shows a processing and sensor interface module 201 and external sensors 232, 234 connected to the module 201. In the illustrated example, the module 201 includes a processor 202, a wireless transceiver 207 having a receiver 206 and a transmitter 209, a memory 210, a first sensor interface 212, a second sensor interface 214, a third sensor interface 216, and an internal sensor 236 connected to the third sensor interface 216. The first and second sensor interfaces 212 and 214 are connected to the first and second external sensors 232, 234 via first and second connection ports 222, 224, respectively. In certain embodiments, some or all of the aforementioned components of the module 201 and other components are mounted on a PCB.

Each of the sensor interfaces 212, 214, 216 can include one or more electronic components that are configured to generate an excitation signal or provide DC power for the sensor that the interface is connected to and/or to condition and digitize a sensor signal from the sensor. For example, the sensor interface can include a signal generator for generating an excitation signal or a voltage regulator for providing power to the sensor. The sensor interface can further include an amplifier for amplifying a sensor signal from the sensor and an analog-to-digital converter for digitizing the amplified sensor signal. The sensor interface can further include a filter (e.g., a low-pass or bandpass filter) for filtering out spurious noises (e.g., a 60 Hz noise pickup).

The processor 202 is configured to send and receive data (e.g., digitized signal or control data) to and from the sensor interfaces 212, 214, 216 via a bus 204, which can be one or more wire traces on the PCB. Although a bus communication topology is used in this embodiment, some or all communication between discrete components can also be implemented as direct links without departing from the scope of the present disclosure. For example, the processor 202 may send data representative of an excitation signal to the sensor excitation signal generator inside the sensor interface and receive data representative of the sensor signal from the sensor interface, over either a bus or direct data links between processor 202 and each of sensor interface 212, 214, and 216.

The processor 202 is also capable of communication with the receiver 206 and the transmitter 209 of the wireless transceiver 207 via the bus 204. For example, the processor 202 using the transmitter and receiver 209, 206 can transmit and receive data to and from the bridge 40. In certain embodiments, the transmitter 209 includes one or more of a RF signal generator (e.g., an oscillator), a modulator (a mixer), and a transmitting antenna; and the receiver 206 includes a demodulator (a mixer) and a receiving antenna which may or may not be the same as the transmitting antenna. In some embodiments, the transmitter 209 may include a digital-to-analog converter configured to receive data from the processor 202 and to generate a base signal; and/or the receiver 206 may include an analog-to-digital converter configured to digitize a demodulated base signal and output a stream of digitized data to the processor 202. In other embodiments, the radio may comprise a direct sequence radio, a software-defined radio, or an impulse spread spectrum radio.

The processor 202 may include a general-purpose processor or a specific-purpose processor for executing instructions and may further include a memory 219, such as a volatile or non-volatile memory, for storing data and/or instructions for software programs. The instructions, which may be stored in a memory 219 and/or 210, may be executed by the processor 202 to control and manage the wireless transceiver 207, the sensor interfaces 212, 214, 216, as well as provide other communication and processing functions.

The processor 202 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable device or a combination of devices that can perform calculations or other manipulations of information.

Information, such as program instructions, data representative of sensor readings, preset alarm conditions, threshold limits, may be stored in a computer or processor readable medium such as a memory internal to the processor 202 (e.g., the memory 219) or a memory external to the processor 202 (e.g., the memory 210), such as a Random Access Memory (RAM), a flash memory, a Read Only Memory (ROM), a Programmable Read-Only Memory (PROM), an Erasable PROM (EPROM), registers, a hard disk, a removable disk, or any other suitable storage device.

In certain embodiments, the internal sensor 236 can be one or more sensors configured to measure certain properties of the processing and sensor interface module 201, such as a board temperature sensor thermally coupled to a PCB. In other embodiments, the internal sensor 236 can be one or more sensors configured to measure certain properties of the patient 10, such as a motion sensor (e.g., an accelerometer) for measuring the patient's motion or position with respect to gravity.

The external sensors 232, 234 can include sensors and sensing arrangements that are configured to produce a signal representative of one or more vital signs of the patient to which the monitor patch 20 is attached. For example, the first external sensor 232 can be a set of sensing electrodes that are affixed to an exterior surface of the monitor patch 20 and configured to be in contact with the patient for measuring the patient's respiratory rate, and the second external sensor 234 can include a temperature sensing element (e.g., a thermocouple or a thermistor or resistive thermal device (RTD)) affixed, either directly or via an interposing layer, to skin of the patient 10 for measuring the patient's body temperature. In other embodiments, one or more of the external sensors 232, 234 or one or more additional external sensors can measure other vital signs of the patient, such as blood pressure, pulse rate, or oxygen saturation.

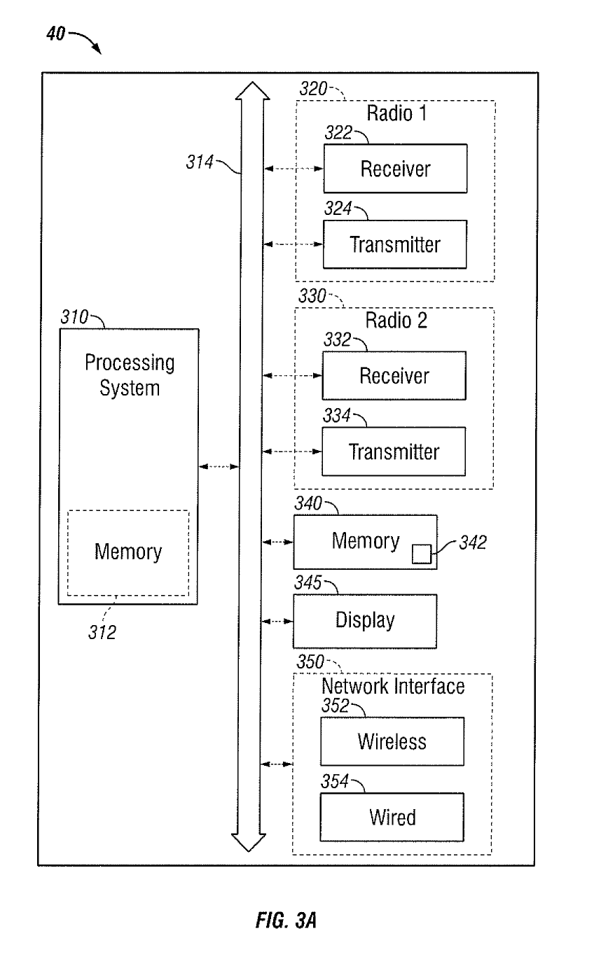

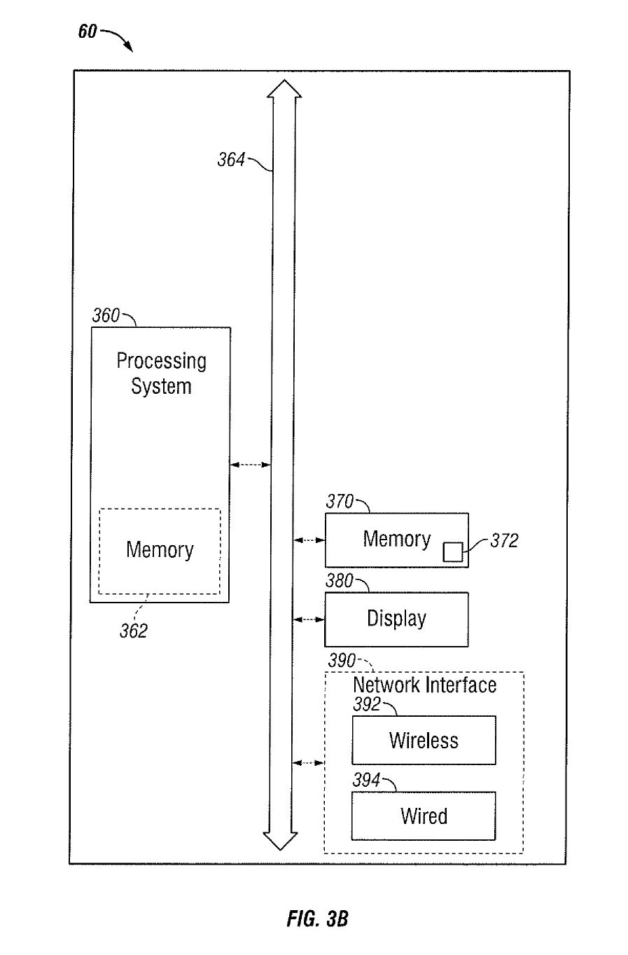

FIG. 3A is a functional block diagram illustrating exemplary electronic components of bridge 40 of FIG. 1 according to one aspect of the subject disclosure. Bridge 40 includes a processor 310, radio 320 having a receiver 322 and a transmitter 324, radio 330 having a receiver 332 and a transmitter 334, memory 340, display 345, and network interface 350 having a wireless interface 352 and a wired interface 354. In some embodiments, some or all of the aforementioned components of module 300 may be integrated into single devices or mounted on PCBs.

Processor 310 is configured to send data to and receive data from receiver 322 and transmitter 324 of radio 320, receiver 332 and transmitter 334 of radio 330 and wireless interface 352 and wired interface 354 of network interface 350 via bus 314. In certain embodiments, transmitters 324 and 334 may include a radio frequency signal generator (oscillator), a modulator, and a transmitting antenna, and the receivers 322 and 332 may include a demodulator and antenna which may or may not be the same as the transmitting antenna of the radio. In some embodiments, transmitters 324 and 334 may include a digital-to-analog converter configured to convert data received from processor 310 and to generate a base signal, while receivers 322 and 332 may include analog-to-digital converters configured to convert a demodulated base signal and sent a digitized data stream to processor 310.

Processor 310 may include a general-purpose processor or a specific-purpose processor for executing instructions and may further include a memory 312, such as a volatile or non-volatile memory, for storing data and/or instructions for software programs. The instructions, which may be stored in memories 312 or 340, may be executed by the processor 310 to control and manage the transceivers 320, 330, and 350 as well as provide other communication and processing functions.

Processor 310 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable device or a combination of devices that can perform calculations or other manipulations of information.

Information such as data representative of sensor readings may be stored in memory 312 internal to processor 310 or in memory 340 external to processor 310 which may be a Random Access Memory (RAM), flash memory, Read Only Memory (ROM), Programmable Read Only Memory (PROM), Erasable Programmable Read Only Memory (EPROM), registers, a hard disk, a removable disk, a Solid State Memory (SSD), or any other suitable storage device.

Memory 312 or 340 can also store a list or a database of established communication links and their corresponding characteristics (e.g., signal levels) between the bridge 40 and its related monitor patches 20. In the illustrated example of FIG. 3A, the memory 340 external to the processor 310 includes such a database 342; alternatively, the memory 312 internal to the processor 310 may include such a database.

FIG. 3B is a functional block diagram illustrating exemplary electronic components of server 60 of FIG. 1 according to one aspect of the subject disclosure. Server 60 includes a processor 360, memory 370, display 380, and network interface 390 having a wireless interface 392 and a wired interface 394. Processor 360 may include a general-purpose processor or a specific-purpose processor for executing instructions and may further include a memory 362, such as a volatile or non-volatile memory, for storing data and/or instructions for software programs. The instructions, which may be stored in memories 362 or 370, may be executed by the processor 360 to control and manage the wireless and wired network interfaces 392, 394 as well as provide other communication and processing functions.

Processor 360 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable device or a combination of devices that can perform calculations or other manipulations of information.

Information such as data representative of sensor readings may be stored in memory 362 internal to processor 360 or in memory 370 external to processor 360 which may be a Random Access Memory (RAM), flash memory, Read Only Memory (ROM), Programmable Read Only Memory (PROM), Erasable Programmable Read Only Memory (EPROM), registers, a hard disk, a removable disk, a Solid State Memory (SSD), or any other suitable storage device.

Memory 362 or 370 can also store a database of communication links and their corresponding characteristics (e.g., signal levels) between monitor patches 20 and bridges 40. In the illustrated example of FIG. 3B, the memory 370 external to the processor 360 includes such a database 372; alternatively, the memory 362 internal to the processor 360 may include such a database.

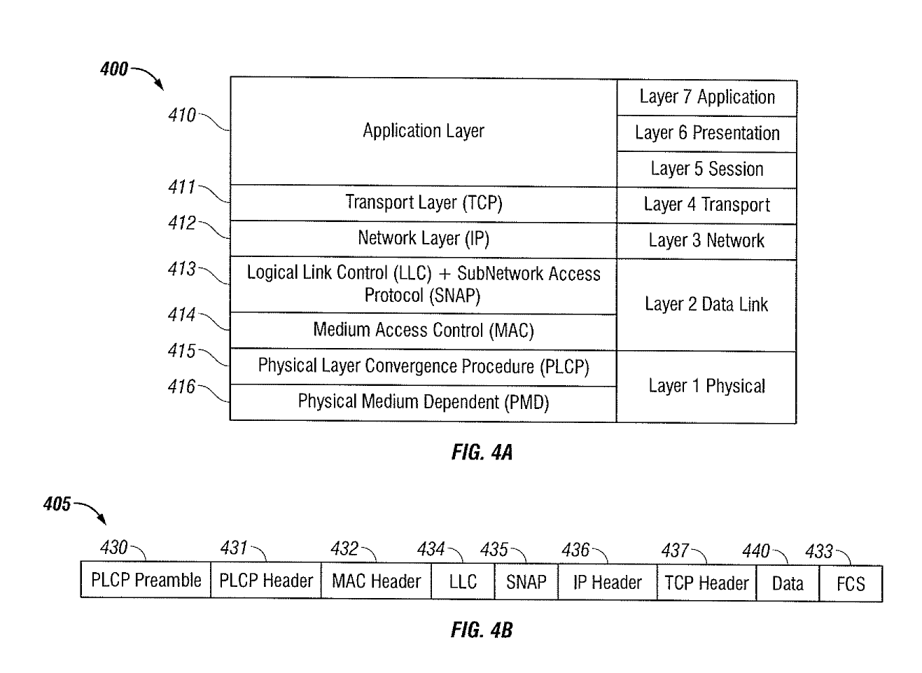

FIGS. 4A & 4B illustrate Ethernet communication protocol 802.11b and an associated message structure. Within this disclosure, the term ‘byte’ will be presumed to be an 8-bit data element. The term ‘packet’ refers to the entire transmitted signal while the term ‘frame’ refers to the structure of the packet.

Ethernet follows the Open System Interconnection Reference Model (OSI Reference Model or OSI Model) shown in FIG. 4A, which is an abstract description for layered communications and computer network protocol design, shown as communication stack 400. The top layer is application layer 410 that generates the data to be transported from one device to another device. To transport a packet of data, the data packet is passed down through layers 411-416. Each layer processes the data packet that is passed to the layer and adds a header of information that is needed to handle the message, then passes the new and larger packet to the layer below it. The Physical Layer is composed of two sublayers 415 and 416. Layer 416 actually transmits the message to the bottom layer of the receiver. At the receiver, the message is passed back up the stack, each layer stripping off the appropriate header. The Institute of Electrical and Electronics Engineers (IEEE) has issued standards for computer communication. Standard IEEE 802.11 is a collection of IEEE standards defining the protocols of each layer for wireless Ethernet.

IEEE 802.11 defines a series of protocols, collectively referred to as “Ethernet”, that use a frame format to define the sequential placement of headers and data in a message. FIG. 4B illustrates the frame for an example message 405 configured to be sent over a 802.11b wireless system directly from one device to another device. The preamble 430 includes a synchronization element (not shown) that is a sequence of alternating zeros and ones and a Start-of-Frame delimiter (not shown) that consists of a defined 16-bit pattern of zeros and ones that enables the receiver to synchronize with the message. The MAC Header 432 contains the addresses of the transmitting and receiving devices, a sequence control number, and other message information. The MAC Layer 414 also adds a Frame Check Sequence (FCS) number 433 which is frequently a Cyclic Redundancy Check (CRC) value. A CRC value can be used with a defined algorithm to provide a reasonable level of assurance that the message has not been corrupted in transit. While it is possible for a message to have errors and still pass the CRC check, successful execution of the CRC algorithm is usually considered sufficient to verify that the message has arrived intact. The remaining headers 434-437 are added by layers 411-416, in reverse order, of stack 400. The data that was generated by application layer 410 is data field 440. Everything in message 405 except data field 440 is ‘overhead’ that is added to transport the data in data field 440 from one device to another device. The lengths of the various overhead fields are: preamble 430 (18 bytes), PLCP Header 431 (6 bytes), MAC Header 432 (18 bytes), LLC 434 (4 bytes), SNAP 435 (5 bytes), IP Header 436 (24 bytes), TCP Header 437 (24 bytes), and FCS 433 (4 bytes), which sums to a total overhead of 103 bytes. It can be seen that sending a few bytes of data in data field 440 carries a very large relative overhead if one is transmitting on a system that follows the 802.11b protocols.

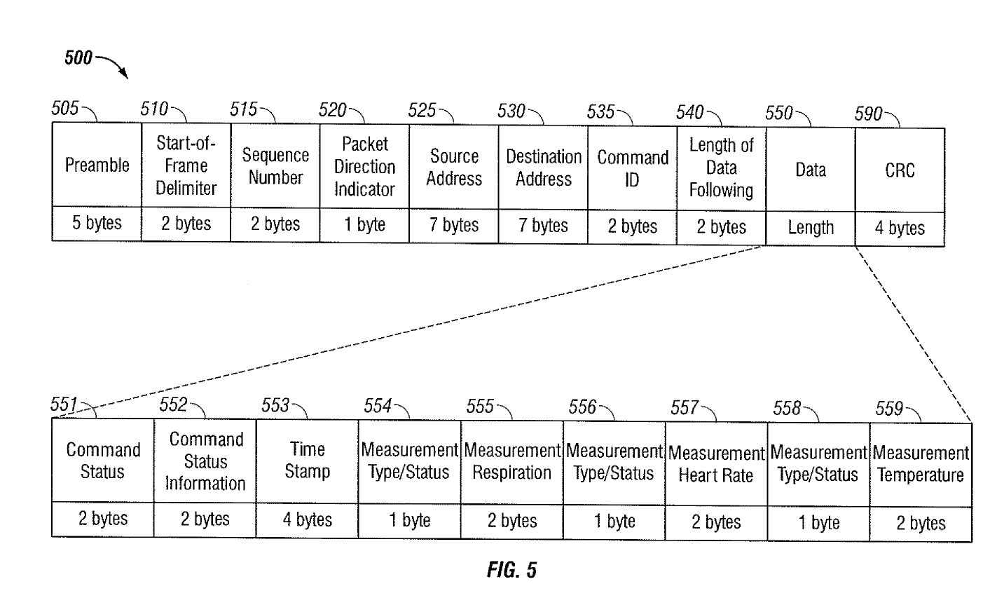

FIG. 5 discloses an example of a communication protocol according to certain aspects of the subject disclosure. The message 500 shown in FIG. 5 is an example of certain embodiments.

Message 500 has a header comprising fields 505, 510, 515, 520, 525, 530, 535, and 540 that contain information enabling the receiver of the transmitted message to synchronize its signal processing with the incoming message, information about the source and destination of the message, information identifying the command, and information related to the amount of data contained in the message. Field 505 is a preamble comprising a sequence of alternating 1s and 0s to establish the signal timing of the message. Field 510 is a start-of-frame field with a fixed configuration of 1s and 0s that is known to the receiver in the patient monitoring system and enables the receiver to detect the start of the actual message. Field 515 contains a sequence number that is incremented for every new message and can be repeated if a message is resent. Field 520 is a packet direction indicator and can be defined to indicate if this message is a bridge-to-patch message or a patch-to-bridge message. Fields 525 and 530 are the addresses, as defined within the patient monitoring system, of the device sending the message and the intended destination device. Field 535 is a command identification field that defines the action to be taken by the patch. Field 540 contains information on the length of the data segment of the message, which may be zero.

The combination of fields 520 and 535 enable the use of a ‘command and response’ protocol wherein the bridge initiates every communication exchange with a message. The bridge sends a message with field 520 configured to indicate that this message is a bridge-to-patch message and a command identified in field 535. The patch whose address matches the destination address in field 530 responds with a message to the bridge that sent the previous message with the same command identification value in field 535 but with field 520 configured to indicate that this is a packet-to-bridge message. In this manner, patches transmit only when commanded to do so by a bridge. In this embodiment, the sizes of each field are as listed in FIG. 5 totaling 28 bytes for the entire header (compared to 103 bytes for conventional Ethernet packet overhead).

The data segment of message 500 comprises fields 551, 552, 553, 554, 555, 556, 557, 558, and 559. This is an exemplary configuration of a data segment that can have more or fewer fields without departing from the scope of this disclosure. Fields 551 and 552 are command status and command status information that may contain, for example, status information related to the amount of measurement data currently stored in memory, remaining battery life, current limits set in the firmware, or the version of firmware currently loaded into the patch memory. The data is contained in pairs of fields, where the first field of each pair is a measurement type field indicating what type measurement is being reported and the second field of each pair is a measurement data field containing the measurement itself In this example, field 554 contains the label indicating that the next measurement is of the respiration rate and field 555 contains the respiration rate measurement. Similarly, field 556 indicates that the next measurement is of the heart rate and field 557 is the heart rate measurement, and field 558 indicates that the next measurement is of body temperature and field 559 is the temperature measurement. Field 553 is a time stamp that is related to, in this example, when the measurement data of fields 555, 557, and 559 was taken. The field sizes, in this embodiment, are 1 byte each for the measurement type fields and 2 bytes each for the measurement data fields.

The final segment of message 500 is the data integrity check value field 590. In this example, a cyclic redundancy check (CRC) value is used to verify that there is a low probability that a message has been corrupted in transmission. While a CRC validation check is efficient and will detect most bit errors in transmitted messages, alternate error-checking protocols will be known to those of ordinary skill in the art and may be substituted without departing from the scope of this disclosure.

A value in using a message protocol such as disclosed in FIG. 5 is that it is shorter than the equivalent message would be if the same amount of data were transmitted using a standard computer communication protocol such as the Ethernet structure depicted in FIG. 4. The benefit is greatest when the amount of data being transferred is small compared to the header and CRC fields. The example message 500 depicted in FIG. 5 will be approximately 50% of the length of the equivalent message configured according to the 802.11b standard. This reduction in message length produces an equivalent reduction in the amount of time that a patch 20 takes to transmit a message, reducing the time that the patch 20 must remain in its ‘awake’ state and consuming power at a higher level than patch 20 consumes while in its ‘sleep’ state.

The use of the protocol of the present disclosure is particularly advantageous when all aspects of a communication system are controlled within a proprietary space. As such, the communication between the patch 20 and bridge 40 can be configured according to this disclosed protocol while the communication between the bridge 40 and server 60 can be, in some embodiments, conducted over a standard Ethernet network and must follow the Ethernet protocols. The benefits of using the disclosed protocol of FIG. 5 accrue to a battery-powered device such as patch 20, as the power saving contributes towards extending the operating life of patch 20. Bridge 40 enables the use of the disclosed protocol for patch 20 as the bridge 40 performs a protocol-conversion to accept data from patch 20 using the disclosed protocol and send the same data to server 60 using, for example, an Ethernet protocol. Similarly, bridge 40 receives messages intended for a patch 20 from server 60 in Ethernet protocol, converts these to the disclosed protocol, and transmits them to patch 20 using the disclosed protocol.

Conserving battery power in wireless devices is one approach to extending the useful life of the wireless device. The patch 20 in the disclosed patient monitoring system can be worn for several days and minimizing the size of the battery reduces the physical impact that wearing a patch 20 has on patient 10. In certain embodiments, patch 20 remains in its ‘awake’ state only long enough to respond to messages from bridge 40 and reducing the length of time that it takes to transmit a message can significantly reduce the duration of this ‘awake’ period.

It can be seen that the disclosed embodiments of the vital-signs monitor patch provide a mobile solution to monitoring the vital signs of a patient. The design of the vital-signs monitor patch frees nurses, or other caregivers, from the task of repetitively measuring the vital signs of their patients, allowing the caregivers to spend more time on other duties. The ability to continuously monitor a patient's vital signs using a monitor patch, together with the rest of the patient monitoring system, increases the ability of the nurse to respond quickly to a sudden change in a patient's condition, resulting in improved care for the patient.

The protocol conversion capability of the bridge 40 enables the use of an advantageous protocol for communication between a patch 20 and bridge 40 while also enabling the use of a standard network using a standard protocol such as Ethernet for communication between bridge 40 and server 60. The use of a standard network reduces the implementation costs and eliminates the need for infrastructure modifications required to run proprietary communication lines between bridges 40 and server 60. As the length of a message using the disclosed protocol can be 50% less than the equivalent message in a standard communication protocol such as Ethernet, the power saving and therefore the useful life of the patch 20 will be increased.

The previous description is provided to enable any person skilled in the art to practice the various aspects described herein. While the foregoing has described what are considered to be the best mode and/or other examples, it is understood that various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. A phrase such an embodiment may refer to one or more embodiments and vice versa.

The word “exemplary” is used herein to mean “serving as an example or illustration.” Any aspect or design described herein as “exemplary” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C.§112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

What is claimed is:

1. A vital-signs patch in a patient monitoring system, the patch comprising a housing configured to be attached to the skin of a patient, the housing containing:
monitoring circuitry configured to acquire and store measurements of vital signs of the patient;
a wireless transmitter configured to transmit signals to another device;
a wireless receiver configured to receive signals from the other device; and
a processor operably connected to the monitoring circuitry, transmitter, and receiver, configured to, upon receipt of an upload signal from the other device, send a message to the other device via the transmitter with a message packet structure comprising:
a data payload wherein the size of the data payload is variable;
a header containing transmit and route information and data payload length; and
a data integrity check value.

2. The vital-signs patch of claim 1 wherein the data being transferred is related to measurements of at least one vital sign of the set of body temperature, cardiac pulse rate, respiration rate, blood pressure, and oxygen saturation.

3. The vital-signs patch of claim 1 wherein the data payload comprises:
a time of measurement; and
one or more measurement data fields;
wherein the measurements are all related to the time of measurement, and wherein the total size of each set of measurement data field and the measurement type field is less than or equal to 3 bytes.

4. The vital-signs patch of claim 3 wherein the data payload further comprises a field associated with each measurement data field indicating the type of measurement.

5. The vital-signs patch of claim 1 wherein the message header comprises:
a preamble comprising a series of alternating 1s and 0s;
a start-of-frame delimiter of a fixed configuration of 1s and 0s;
a sequence number, wherein the sequence number of a message is used again for a retransmission of that message and is incremented for each subsequent new message; and
a command identification value, wherein command identification values may be associated with a data payload length of zero;
wherein the size of the message header is less than 30 bytes.

6. The vital-signs patch of claim 1 wherein the data integrity check value comprises a cyclic redundancy check value.

7. The vital-signs patch of claim 1 wherein the message packet structure comprises:
preamble, 5 bytes;
start-of-frame delimiter field, 2 bytes;
message sequence number field, 2 bytes;
packet direction indicator field, 1 byte;
source address field, 7 bytes;
destination address field, 7 bytes;
command identification field, 2 bytes;
length of data field, 2 bytes;
data—command status field, 2 bytes;
data—command status information field, 2 bytes;
data—time stamp field, 4 bytes;
one or more pairs of data fields, wherein each pair comprises:
measurement type/status field, 1 byte; and
measurement data field, 2 bytes; and
a CRC field, 4 bytes;
wherein the message packet comprises one or more time stamp fields each followed by pairs of measurement type/status and measurement data fields.

8. A patient monitoring system, comprising:
a vital-signs patch configured to be attached to the skin of a patient, and further configured to communicate wirelessly;
a bridge configured to communicate with the patch;
wherein the patch and bridge are configured to exchange messages using a message packet structure comprising:
a data payload, wherein the size of the data payload is variable;
a header containing the information necessary to transmit and route the message, and the length of the data payload; and
a data integrity check value that can be used with a defined message inspection protocol to verify that the message has been received intact; and
wherein the bridge is configured to initiate each exchange with a command message and the patch is configured to respond with an acknowledgement message.

9. The patient monitoring system of claim 8 wherein the patch is configured to make, store, and transmit measurements of at least one vital sign of the set of body temperature, cardiac pulse rate, respiration rate, blood pressure, and oxygen saturation.

10. The patient monitoring system of claim 8 wherein the data payload comprises:
a time of measurement field;
one or more measurement data fields; and
a field associated with each measurement data field indicating the type of measurement;
wherein the measurements contained in a message are all gathered approximately at the time specified in the time of measurement field, and wherein the total size of each set of measurement data field and the measurement type field is less than or equal to 3 bytes.

11. The patient monitoring system of claim 8 wherein the header field comprises:
a preamble comprising a series of alternating 1s and 0s;
a start-of-frame delimiter of a fixed configuration of 1s and 0s;
a sequence number, wherein the sequence number of a message is used again for a retransmission of that message and is incremented for each subsequent new message; and
a command identification value, wherein command identification values may be associated with a data payload length of zero;
wherein the size of the message header is less than 30 bytes.

12. The patient monitoring system of claim 8 wherein the data integrity check value comprises a cyclic redundancy check value.

13. The patient monitoring system of claim 8 wherein the message packet structure comprises:
preamble, 5 bytes;
start-of-frame delimiter field, 2 bytes;
message sequence number field, 2 bytes;
packet direction indicator field, 1 byte;
source address field, 7 bytes;
destination address field, 7 bytes;
command identification field, 2 bytes;
length of data field, 2 bytes;
data—command status field, 2 bytes;
data—command status information field, 2 bytes;
data—time stamp field, 4 bytes;
one or more pairs of data fields, wherein each pair comprises:
measurement type/status field, 1 byte; and
measurement data field, 2 bytes; and
a CRC field, 4 bytes;
wherein the message packet comprises one or more time stamp fields each followed by one or more pairs of measurement type/status and measurement data fields.

14. A method of conserving battery power in a patch having vital-signs monitoring circuitry in a patient monitoring system, comprising the steps of:
receiving a transmit signal;
retrieving vital-signs data;
creating a message that contains:
a data payload that contains at least a portion of the vital-signs data retrieved, wherein the size of the data payload is variable and may be zero;
a header containing the information necessary to transmit and route the message, and the length of the data payload; and
a data integrity check value that can be used with a defined message inspection protocol to verify that the message has been received intact; and
transmitting the message.

15. The method of claim 13 wherein the header field comprises:
a preamble comprising a series of alternating 1s and 0s;
a start-of-frame delimiter of a fixed configuration of 1s and 0s;
a sequence number, wherein the sequence number of a message is used again for a retransmission of that message and is incremented for each subsequent new message; and
a command identification value, wherein command identification values may be associated with a data payload length of zero;
wherein the size of the message header is less than 30 bytes.

16. The method of claim 13 wherein the data payload comprises:
a time of measurement field;
one or more measurement data fields; and
a field associated with each measurement data field indicating the type of measurement;
wherein the measurements contained in a message are all gathered approximately at the time specified in the time of measurement field, and wherein the total size of each set of measurement data field and the measurement type field is less than or equal to 3 bytes.

17. The method of claim 13 wherein the data integrity check value comprises a cyclic redundancy check value.

18. The method of claim 13 wherein the message packet structure comprises:
preamble, 5 bytes;
start-of-frame delimiter field, 2 bytes;
message sequence number field, 2 bytes;
packet direction indicator field, 1 byte;
